(19) United States
(12) Patent Application Publication
(10) Pub. No.: US 2024/0425460 A1
(43) Pub. Date: Dec. 26, 2024

(54) CRYSTAL FORM OF NIROGACESTAT DIHYDROBROMIDE, AND PREPARATION METHOD THEREFOR, AND USE THEREOF

(71) Applicant: CRYSTAL PHARMACEUTICAL (SUZHOU) CO., LTD., Suzhou (CN)

(72) Inventors: Yinan SHEN, Suzhou (CN); Jiaming SHI, Suzhou (CN); Liping MENG, Suzhou (CN)

(73) Assignee: CRYSTAL PHARMACEUTICAL (SUZHOU) CO., LTD., Suzhou (CN)

(21) Appl. No.: 18/822,448

(22) Filed: Sep. 2, 2024

Related U.S. Application Data
(63) Continuation of application No. PCT/CN2023/082030, filed on Mar. 17, 2023.

(30) Foreign Application Priority Data
Mar. 17, 2022 (CN) 202210265903.7
Apr. 29, 2022 (CN) 202210473159.X

(51) Int. Cl. C07D 233/88; A61K 9/20; A61K 31/417

(52) U.S. Cl. CPC C07D 233/88; A61K 9/2013; A61K 9/2018; A61K 9/2027; A61K 9/2054; A61K 9/2095; A61K 31/417

(57) ABSTRACT

Provided are novel crystalline forms of Nirogacestat (Referred to as “Compound I”) dihydrobromide and preparation methods thereof, pharmaceutical compositions containing the crystalline forms, and uses of the crystalline forms for preparing γ-secretase inhibitor drugs and drugs for treating desmoid tumors. Compared with prior arts, the provided crystalline forms of Compound I dihydrobromide have one or more improved properties, which solve the problems of the prior art and are of great value to the optimization and development of the drugs.

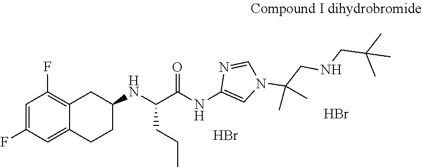 [SMILES: CCC[C@H](N[C@H]1CCc2cc(F)cc(F)c2C1)C(=O)Nc1cn(C(C)(C)CNCC(C)(C)C)cn1 ; Br ; Br]

### CROSS-REFERENCE TO RELATED APPLICATIONS

This application is a continuation of International Patent Application No. PCT/CN2023/082030, filed on Mar. 17, 2023, which claims priority to Chinese Patent Application No. 202210265903.7 and No. 202210473159.X, filed on Mar. 17, 2022 and Apr. 29, 2022, respectively, the entire contents of which are incorporated herein by reference.

### TECHNICAL FIELD

The present disclosure pertains to the field of chemical crystallography, particularly relates to novel crystalline forms of Nirogacestat dihydrobromide, preparation method and use thereof.

### BACKGROUND

Desmoid tumors, also known as aggressive fibromatosis, are rare locally invasive and slow-growing soft tissue tumors. Despite its inability to metastasize and its classification as a benign tumor, desmoid tumor can still lead to serious complications and occasionally mortality in patients. Therefore, there is an urgent need for drug treating desmoid tumors.

A promising development is the γ-secretase inhibitor Nirogacestat, developed by Spring Works Therapeutics, which has received FDA breakthrough therapy and fast track designation, as well as orphan drug designation from FDA and EMA. Nirogacestat is being investigated for the treatment of desmoid tumors due to its ability to bind to γ-secretase, blocking proteolytic activation of Notch receptors and obtained positive clinical results. Previous clinical study data have shown that Notch signaling plays an important role in cancer development. Hence, inhibiting Notch signaling is an important strategy for treating desmoid tumors.

The chemical name of Nirogacestat is (S)-2-(((S)-6,8-difluoro-1,2,3,4-tetrahydronaphthalen-2-yl) amino)-N-(1-(2-methyl-1-(neopentylamino) propan-2-yl)-1H-imidazol-4-yl) pentanamide (referred to as “Compound I”) and the structure of Compound I dihydrobromide is shown as the follows:

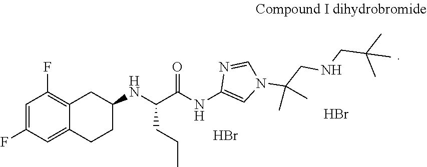 [SMILES: CCC[C@H](N[C@H]1CCc2cc(F)cc(F)c2C1)C(=O)Nc1cn(C(C)(C)CNCC(C)(C)C)cn1 ; Br ; Br]

It is well known in the field that drug polymorphism is a common phenomenon in small molecule drug development and it is an important factor affecting drug quality. A crystalline form is a solid material whose constituents are arranged in a highly ordered microscopic structure, forming a crystal lattice that extends in all directions. Polymorphism refers to the phenomenon that a compound exists in more than one crystalline form. Compounds may exist in one or more crystalline forms, but their existence and characteristics cannot be predicted with any certainty. Different crystalline forms of drug substances have different physicochemical properties, which can affect drug's in vivo dissolution and absorption and will further affect drug's clinical efficacy and safety to some extent. In particular, for some poorly soluble oral solid or semi-solid dosage forms, crystalline forms can be crucial to the performance of drug product. In addition, the physiochemical properties of a crystalline form are very important to the manufacturing process. Therefore, polymorphism is an important part of drug research and drug quality control.

Therefore, in order to obtain crystalline forms with acceptable physicochemical properties (including chemical stability, thermal stability, solubility, hygroscopicity and/or particle size), and manufacturability (including yield, impurity rejection during crystallization, filtration properties, drying properties and milling properties), as well as formulation feasibility (including pressure stability or compression forces stability during tableting). It requires comprehensive research on the crystallization behavior of Compound I dihydrobromide to obtain crystalline forms that meet the pharmaceutical needs of Compound I.

At present, only prior art WO2021029854A1 disclosed crystalline forms of Compound I dihydrobromide. However, the XRPD patterns disclosed in the application show that Form B, Form G, Form H, Form H′, Form K, Form M and Form N contain a large amount of amorphous and have poor crystallinity. The drug substances with high amorphous content will affect its stability, process, storage, hygroscopicity, as well as the dissolution rate of the drug product, making it unsuitable for medicinal use. Furthermore, after in-depth research of WO2021029854A1, it was found that the Form A is the preferred crystalline form with better crystallinity disclosed in prior art. However, the solubility of Form A is low. According to the standards of drug solubility in Chinese Pharmacopoeia and the United States Pharmacopoeia, the solubility of Form A in water belongs to slightly soluble. Form A has low solubility in water, which is not beneficial to drug's in vivo absorption, thereby affecting the bioavailability of the drug. In addition, inventors of the present disclosure prepared Form A according to the method disclosed in prior art WO2021029854A1. Further research found that the particle size distribution of Form A was non-uniform and Form A are easy to agglomerate.

To overcome the disadvantages of prior arts, a novel crystalline form meeting the medicinal standards is still needed for the development of drugs containing Compound I dihydrobromide. However, prior art WO2021029854A1 has disclosed multiple crystalline forms of Compound I dihydrobromide, it is not easy to obtain a novel crystalline form which can overcome the disadvantages of the prior arts as well as meeting medicinal standards. In order to avoid the methods used in the prior art, inventors of the present disclosure designed nearly thousands of experiments and tried various experimental methods such as evaporation, slurry, gas-liquid diffusion, gas-solid diffusion, cooling and humidity induction, but no novel crystalline forms were obtained. Finally, inventors of the present disclosure surprisingly obtained anhydrous forms of Compound I dihydrobromide in the present disclosure in an unconventional organic solvent trifluoroethanol through an unconventional method, which have advantages in at least one aspect of solubility, hygroscopicity, purification ability, stability, adhesiveness, compressibility, flowability, in vitro and in vivo dissolution, and bioavailability, etc. In particular, the crystalline forms of the Compound I dihydrochloride of the present disclosure have advantages such as better solubility, uniform particle size distribution, no agglomeration, no solvent residue, better flowability, high density, better compressibility and good stability of drug substances and drug product, which solve the problems existing in prior arts and are of great significance for the development of drugs containing Compound I dihydrobromide.

### SUMMARY

The present disclosure is to provide novel crystalline forms of Compound I dihydrobromide, preparation method and pharmaceutical compositions comprising the crystalline forms.

According to the objective of the present disclosure, crystalline form CSII of Compound I dihydrobromide is provided by the present disclosure (hereinafter referred to as Form CSII).

In one aspect provided herein, the X-ray powder diffraction pattern of Form CSII comprises characteristic peaks at 2theta values of 7.6°±0.2°, 8.4° 0.2° and 12.0°±0.2° using CuKα radiation.

Furthermore, the X-ray powder diffraction pattern of Form CSII comprises one or two or three characteristic peaks at 2theta values of 18.5°±0.2°, 19.5°±0.2° and 20.5°±0.2° using CuKα radiation. Preferably, the X-ray powder diffraction pattern of Form CSII comprises characteristic peaks at 2theta values of 18.5°±0.2°, 19.5°±0.2° and 20.5°±0.2° using CuKα radiation.

In another aspect provided herein, the X-ray powder diffraction pattern of Form CSII comprises three or four or five or six or seven or eight or nine or ten characteristic peaks at 2theta values of 7.6°±0.2°, 8.4°±0.2°, 12.0°±0.2°, 18.5°±0.2°, 19.5°±0.2°, 20.5°±0.2°, 15.3°±0.2°, 16.9°±0.2°, 24.1°±0.2° and 27.1°±0.2° using CuKα radiation.

Without any limitation being implied, an XRPD pattern of Form CSII is substantially as depicted in FIG. 1.

Without any limitation being implied, a TGA curve of Form CSII is substantially as depicted in FIG. 2, which shows 1.0% weight loss when heated to 100° C.

Without any limitation being implied, Form CSII is an anhydrate.

According to the objective of the present disclosure, a process for preparing Form CSII is also provided. The process comprises:

Dissolving Compound I dihydrobromide into trifluoroethanol, evaporating to obtain a solid form and then heating the solid form to obtain Form CSII.

Furthermore, said heating temperature is preferably 120° C. to 200° C., further preferably 150° C. Said heating time is preferably 0.5 min to 60 min.

According to the objective of the present disclosure, crystalline form CSIII of Compound I dihydrobromide is provided by the present disclosure (hereinafter referred to as Form CSIII).

In one aspect provided herein, the X-ray powder diffraction pattern of Form CSIII comprises characteristic peaks at 2theta values of 8.4°±0.2°, 8.8°±0.2° and 16.3°±0.2° using CuKα radiation.

Furthermore, the X-ray powder diffraction pattern of Form CSIII comprises one or two or three characteristic peaks at 2theta values of 12.7°±0.2°, 14.1° 0.2° and 20.8°±0.2° using CuKα radiation. Preferably, the X-ray powder diffraction pattern of Form CSIII comprises characteristic peaks at 2theta values of 12.7°±0.2°, 14.1°±0.2° and 20.8°±0.2° using CuKα radiation.

Furthermore, the X-ray powder diffraction pattern of Form CSIII comprises one or two characteristic peaks at 2theta values of 21.7°±0.2° and 22.2°±0.2° using CuKα radiation. Preferably, the X-ray powder diffraction pattern of Form CSIII comprises characteristic peaks at 2theta values of 21.7°±0.2° and 22.2°±0.2° using CuKα radiation.

In another aspect provided herein, the X-ray powder diffraction pattern of Form CSIII comprises three or four or five or six or seven or eight characteristic peaks at 2theta values of 8.4°±0.2°, 8.8°±0.2°, 16.3°±0.2°, 12.7°±0.2°, 14.1°±0.2°, 20.8°±0.2°, 21.7°±0.2° and 22.2°±0.2° using CuKα radiation.

Without any limitation being implied, an XRPD pattern of Form CSIII is substantially as depicted in FIG. 3.

Without any limitation being implied, a TGA curve of Form CSIII is substantially as depicted in FIG. 5, which shows 0.8% weight loss when heated to 100° C.

Without any limitation being implied, Form CSIII is an anhydrate.

According to the objective of the present disclosure, a process for preparing Form CSIII is also provided. The process comprises:

Dissolving Compound I dihydrobromide into trifluoroethanol, evaporating to obtain a solid form and then heating the solid form with specific heating rate to obtain Form CSIII.

Furthermore, said heating rate is preferably 1° C./min to 5° C./min, further preferably 2° C./min. Said heating temperature is preferably 201° C. to 205° C., further preferably 202° C.

According to the objective of the present disclosure, the present disclosure also provides the use of Form CSII, Form CSIII or combinations thereof for preparing other crystalline forms, salts or co-crystals of Compound I.

According to the objective of the present disclosure, a pharmaceutical composition is provided, said pharmaceutical composition comprises a therapeutically effective amount of Form CSII, Form CSIII or combinations thereof and pharmaceutically acceptable excipients.

According to the objective of the present disclosure, Form CSII, Form CSIII or combinations thereof can be used for preparing γ-secretase inhibitor drugs.

According to the objective of the present disclosure, Form CSII, Form CSIII or combinations thereof can be used for preparing drugs treating desmoid tumors.

### Technical Effects and Problems Solved by the Present Disclosure

The technical problem solved by the present disclosure is to provide novel crystalline forms different from the crystalline forms in prior arts. While maintaining excellent stability base, the novel crystalline forms also have higher solubility, more uniform particle size distribution, smaller particle size, better flowability, higher density, better compressibility, no solvent residue and no agglomeration compared with prior art Form A, which solves the problems existing in prior arts.

Form CSII of the present disclosure has the following advantages:

(1) Compared with prior art Form A, Form CSII of the present disclosure has a higher solubility. Particularly in water, the solubility of Form CSII is 3-5 times that of Form A. In FeSSIF and FaSSIF, the solubilities of Form CSII are 1.5 times that of Form A. Higher solubility of Form CSII of the present disclosure is beneficial to improve drug's in vivo absorption and bioavailability. In addition, drug dose reduction without affecting efficacy is possible due to higher solubility, thereby reducing the drug's side effects and improving drug safety.

(2) Compared with prior art Form A, Form CSII of the present disclosure has no agglomeration, and shows a smaller particle size and more uniform particle size distribution. Uniform particle size is beneficial to reduce solvent enrichment, improve the purity of the drug substance, and reduce solvent residue, which ensures uniformity of drug content and reduces variability of in vitro dissolution. Small particle size is beneficial to improve drug's solubility and bioavailability.

(3) Compared with prior art Form A, Form CSII of the present disclosure has higher destiny. Test results indicate that the bulk density and tapped density of Form CSII are remarkably higher than that of prior art Form A. Higher density of Form CSII is beneficial to large scale production. Higher density of Form CSII can also reduce dust and occupational hazard.

(4) Compared with prior art Form A, Form CSII of the present disclosure has higher flowability. Better flowability can prevent clogging of production equipment and increase manufacturing efficiency. Better flowability of Form CSII ensures the content uniformity of drug products, reduces the weight variation of drug products and improves products quality.

(5) Compared with prior art Form A, Form CSII of the present disclosure has better compressibility. Failure in hardness/friability test and tablet crack issue can be avoided due to better compressibility of Form CSII, making the preparation process more reliable, improving product appearance, promoting product quality and production efficiency.

(6) Form CSII of the present disclosure has no solvent residue. Solvent residue will not only affect safety of drugs, but also affect quality and stability of drugs. Solvent residue may cause crystal transformation or impurity generation during the production and storage of drugs, resulting in bioavailability change and toxicity. Form CSII of the present disclosure with no solvent residue effectively overcomes the disadvantages of poor stability, poor efficacy and high toxicity caused by low purity or high solvent residue of drug substance.

(7) Form CSII of the present disclosure has good physical stability under mechanical force. The crystalline form of Form CSII doesn't change after drug manufacturing process. Grinding and pulverization are often required in the drug manufacturing process. Good physical stability of the drug substance can reduce the risk of crystallinity decrease and crystal transformation during the drug production process. Furthermore, Form CSII has good physical stability under 5 kN pressure, which is beneficial to keep crystalline form unchanged during tableting process.

(8) From CSII drug substance and drug product of the present disclosure have good stability. Crystalline state of Form CSII drug substance doesn't change for at least 9 months when stored under the condition of 25° C./60% RH. The chemical purity remains substantially unchanged during storage.

Meanwhile, crystalline state of Form CSII drug substance doesn't change for at least 6 months when stored under the condition of 40° C./75% RH. The chemical purity remains substantially unchanged during storage. After Form CSII is mixed with the excipients to form a drug product and stored under the conditions of 40° C./75% RH and 60° C./75% RH, crystalline state of Form CSII drug product doesn't change for at least one month. These results show that From CSII drug substance and drug products of the present disclosure have good stability under accelerated and stress conditions. Drug substance and drug product would go through high temperature and high humidity conditions caused by different season, regional climate and environment during storage, transportation, and manufacturing processes. Therefore, good stability under accelerated and stress conditions is of great importance to the drug development. Form CSII drug substance and drug product have good stability under stress conditions, which is beneficial to avoid the impact on drug quality due to crystal transformation or decrease in purity during drug storage.

Good physical and chemical stability of drug substance ensure that no crystal transformation or impurities is generated during production and storage. Form CSII has good physical and chemical stability, ensuring consistent and controllable quality of the drug substance and drug product, minimizing quality change, bioavailability change and toxicity due to crystal transformation or impurity generation.

Form CSIII of the present disclosure has the following advantages:

(1) Compared with prior art Form A, Form CSIII of the present disclosure has a higher solubility. Particularly in FeSSIF, the solubility of Form CSIII is 1.5 times that of Form A. In FaSSIF, the solubility of Form CSIII is 1.3 times that of Form A. Higher solubility of Form CSIII drug substance of the present disclosure is beneficial to improve drug's in vivo absorption and bioavailability. In addition, drug dose reduction without affecting efficacy is possible due to higher solubility, thereby reducing the drug's side effects and improving drug safety.

(2) Compared with prior art Form A, Form CSIII of the present disclosure has no agglomeration and more uniform particle size distribution. Uniform particle size is beneficial to reduce solvent enrichment and solvent residue as well as improve the purity of the drug substance. Uniform particle size also helps to ensure uniformity of content and reduce variability of in vitro dissolution.

(3) Form CSIII of the present disclosure has no solvent residue. Solvent residue affects the safety, quality and stability of drug product. Solvent residue may lead to crystal transformation or impurities generation during production and storage, further lead to bioavailability change and toxicity. Form CSIII of the present disclosure has no solvent residue, which effectively overcome the disadvantages of poor stability, poor efficacy and high toxicity caused by the low purity or high solvent residue of drug substances.

(4) From CSIII drug substance of the present disclosure has good stability. Crystalline state of Form CSIII drug substance doesn't change for at least 3 months when stored under the condition of 25° C./60% RH.

Meanwhile, crystalline state of Form CSIII drug substance doesn't change for at least 3 months when stored under the condition of 40° C./75% RH. Crystalline state of Form CSIII drug substance doesn't change for at least 2 months when stored under the condition of 60° C./75% RH. These results show that From CSIII drug substance of the present disclosure has good stability under accelerated and stress conditions. Drug substance and drug product would go through high temperature and high humidity conditions caused by different season, regional climate and environment during storage, transportation, and manufacturing processes. Therefore, good stability under accelerated and stress conditions is of great importance to the drug development. Form CSIII drug substance have good stability under stress condition, which is beneficial to avoid the impact on drug quality due to crystal transformation or decrease in purity during drug storage.

Good physical and chemical stability of drug substance ensure that no crystal transformation or impurities is generated during production and storage. Form CSIII has good physical and chemical stability, ensuring consistent and controllable quality of the drug substance, minimizing quality change, bioavailability change and toxicity due to crystal transformation or impurity generation.

### BRIEF DESCRIPTION OF THE DRAWINGS

FIG. 1 shows an XRPD pattern of Form CSII.

FIG. 2 shows a TGA curve of Form CSII.

FIG. 3 shows an XRPD pattern of Form CSIII.

FIG. 4 shows an XRPD pattern of Form CSIII.

FIG. 5 shows a TGA curve of Form CSIII.

FIG. 6 shows a PLM pattern of Form CSII and prior art Form A (top: prior art Form A, bottom: Form CSII).

FIG. 7 shows a particle size distribution curve of prior art Form A before and after ultrasonication. (top: before ultrasonication, bottom: after ultrasonication).

FIG. 8 shows a particle size distribution curve of Form CSII before and after ultrasonication. (top: before ultrasonication, bottom: after ultrasonication).

FIG. 9 shows an XRPD pattern overlay of Form CSII before and after tableting (from bottom to top: before tableting, after tableting).

FIG. 10 shows an XRPD pattern overlay of Form CSII drug substance before and after storage under different conditions (from bottom to top: initial, 25° C./60% RH for 9 months sealed with desiccants and antioxidants, 40° C./75% RH for 6 months sealed with desiccants and antioxidants).

FIG. 11 shows an XRPD pattern overlay of Form CSII before and after formulation process (from bottom to top: Form CSII drug substance, Form CSII after formulation process, blank mixed powder after formulation process).

FIG. 12 shows an XRPD pattern overlay of Form CSII drug product before and after storage under different conditions (from bottom to top: initial, 40° C./75% RH for one month sealed with desiccants and antioxidants, 60° C./75% RH for one month sealed with desiccants and antioxidants).

FIG. 13 shows a PLM pattern of Form CSIII and prior art Form A (top: prior art Form A, bottom: Form CSIII).

FIG. 14 shows an XRPD pattern overlay of Form CSIII drug substance before and after storage under different conditions (from bottom to top: initial, 25° C./60% RH for 3 months sealed with desiccants and antioxidants, 40° C./75% RH for 3 months sealed with desiccants and antioxidants, 60° C./75% RH for 2 months sealed with desiccants and antioxidants).

FIG. 15 shows an XRPD pattern of Type K13.

FIG. 16 shows an XRPD pattern of Type K13.

FIG. 17 shows an XRPD pattern of Type K13.

### DETAILED DESCRIPTION

The present disclosure is further illustrated by the following examples which describe the preparation and use of the crystalline forms of the present disclosure in detail. It is obvious to those skilled in the art that changes in the materials and methods can be accomplished without departing from the scope of the present disclosure.

The abbreviations used in the present disclosure are explained as follows:
XRPD: X-ray Powder Diffraction
TGA: Thermo Gravimetric Analysis
PLM: Polarized Light Microscopy
PSD: Particle Size Distribution
¹H NMR: Proton Nuclear Magnetic Resonance
HPLC: High Performance Liquid Chromatography
UPLC: Ultra performance liquid chromatography
FeSSIF: Fed State Simulated Intestinal Fluid
FaSSIF: Fasted State Simulated Intestinal Fluid
RH: Relative humidity
HDPE: High Density Polyethylene
LDPE: Low Density Polyethylene
   Instruments and methods used for data collection:

XRPD patterns in the present disclosure were acquired by a Bruker X-ray powder diffractometer. The parameters of the X-ray powder diffraction method of the present disclosure are as follows:
X-Ray source: Cu, Kα
Kα2/Kα1 intensity ratio: 0.50

TGA data in the present disclosure were acquired by a TA Q500. The parameters of the TGA method of the present disclosure are as follows:
Heating rate: 10° C./min
Purge gas: N₂

¹H NMR data were collected from a Bruker Avance II DMX 400M Hz NMR spectrometer. 1-5 mg of sample was weighed and dissolved with 0.5 mL of deuterated dimethyl sulfoxide to obtain a solution with a concentration of 2-10 mg/mL.

PLM data in the present disclosure were acquired by Sunny CX40P polarizing microscope.

The parameter of the PLM method of the present disclosure is as follows:
Magnification times: 100

The particle size distribution data in the present disclosure were acquired by a Mastersizer 3000 laser particle size analyzer of Malvern. The test was carried out in wet mode, using a Hydro MV dispersion device, and the dispersant was Isopar G. The parameters are as follows:

| | |
|---|---|
| Size distribution: Volume | Measurement duration: 10 s |
| Dispersant: Isopar G | Particle coordinates: Standard |
| Number of measurements: 3 | Fluid refractive index: 1.42 |
| Absorption index: 0.100 | Residuals: Enabled |
| Particle refractive index: 1.520 | Speed: 2500 rpm |
| Particle type: Irregular | Ultrasonic power/time: 30 W/30 s |

The parameters of related substance detection in the present disclosure are shown in Table 1.

TABLE 1

| HPLC | Agilent 1260 with DAD/VWD detector | |
|---|---|---|
| Column | ZORBAX Eclipse XDB-C18, 4.6 × 100 | |
| | mm 3.5 μm | |
| Ghost-Buster column | Welch Ghost-Buster, 4.6*50 mm | |
| Mobile phase | A: 10 mM KH₂PO₄ in H₂O (pH 6.0, TEA) | |
| | B: Acetonitrile | |
| Gradient | Time (min) | % A |
| | 0.0 | 80 |
| | 1.0 | 80 |
| | 6.0 | 50 |
| | 19.0 | 20 |
| | 30.0 | 20 |
| | 31.0 | 80 |
| | 40.0 | 80 |

| | |
|---|---|
| Run time | 40 min |
| Flow rate | 1.0 mL/min |
| Injection volume | 5 μL |
| Detector wavelength | 240 nm |
| Column temperature | 40° C. |
| Sample pan temperature | Room temperature |
| Diluent | 50% Acetonitrile |

The parameters of kinetic solubility in the present disclosure are shown in Table 2.

TABLE 2

| UPLC | Agilent 1260 with DAD detector | |
|---|---|---|
| Column | ZORBAX Eclipse XDB-C18, 4.6 × 100 mm | |
| | 3.5 μm | |
| Ghost-Buster column | Welch Ghost-Buster, 4.6 × 50 mm | |
| Mobile phase | A: 0.1% H₃PO₄ in H₂O (pH 6.0, TEA) | |
| | B: Acetonitrile | |
| Isocratic elution | Time (min) | B % |
| | 0.0 | 78 |

| | |
|---|---|
| Run time | 8 min |
| Post time | 0.0 min |
| Flow rate | 1.0 mL/min |
| Injection volume | 5 μL |
| Detector wavelength | 240 nm |
| Column temperature | 40° C. |
| Sampler temperature | Room temperature |
| Diluent | 50% Acetonitrile |

Said “evaporating” is accomplished by using a conventional method in the field such as slow evaporation or rapid evaporation. Slow evaporation is accomplished in a container covered by a sealing film with pinholes. Rapid evaporation is accomplished in an open container.

Said “room temperature” is not a specific temperature, but a temperature range of 10-30° C.

Said “characteristic peak” refers to a representative diffraction peak used to distinguish crystals. The 2theta value of diffraction peak usually can have a deviation of ±0.20 using CuKα radiation.

Said “anhydrate” refers to a solid form that without crystalline water or solvents.

Said “isomorphous solvate” refers to similarity or sameness in chemical compositions and, under the same thermodynamic conditions, the crystal structures are the same. As a result, the XRPD patterns of isomorphous crystals are identical or similar.

In the present disclosure, “crystal” or “crystalline form” refers to the crystal or the crystalline form being identified by the X-ray diffraction pattern shown herein. Those skilled in the art are able to understand that the X-ray powder diffraction pattern depend on the instrument conditions, the sample preparation and the purity of samples. The relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSII and Form CSIII of the present disclosure are pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

Unless otherwise specified, the following examples were conducted at room temperature.

According to the present disclosure, Compound I used as raw materials include, but are not limited to solid (crystalline and amorphous), oil, liquid form or solution. Preferably, Compound I used as the raw material is a solid.

Raw materials of Compound I used in the following examples were prepared by prior arts, for example, the method disclosed in WO2021029854A1.

### Example 1 Preparation of Form CSII

201.3 mg of Compound I dihydrobromide was dissolved into 2.0 mL of trifluoroethanol to obtain a clear solution, and then the solution was evaporated at room temperature for 5 days to obtain a solid. Two appropriate amounts of the obtained solid were heated to 150° C. under nitrogen protection and the temperature was kept at 150° C. for 5 min, then the temperature was decreased to room temperature to obtain crystalline solids. The crystalline solids were marked as sample 1 and sample 2 respectively.

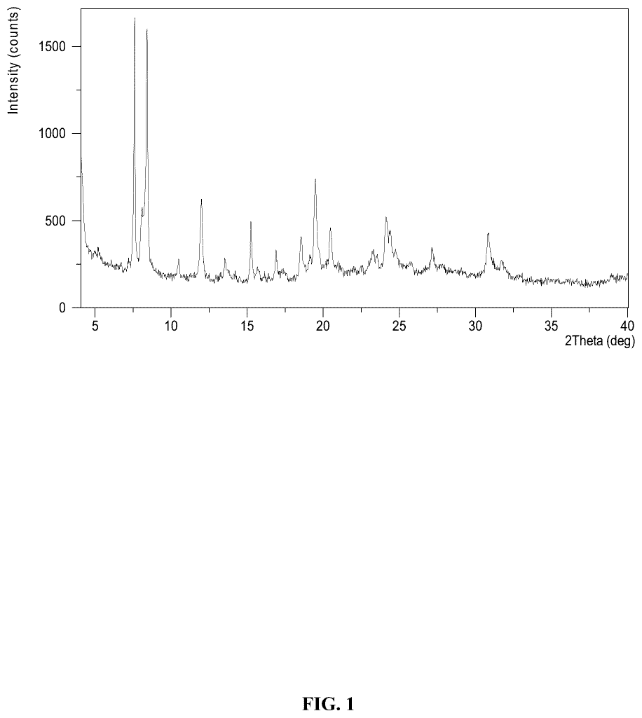

The sample 1 was confirmed to be Form CSII. The XRPD pattern is substantially as depicted in FIG. 1, and the XRPD data are listed in Table 3.

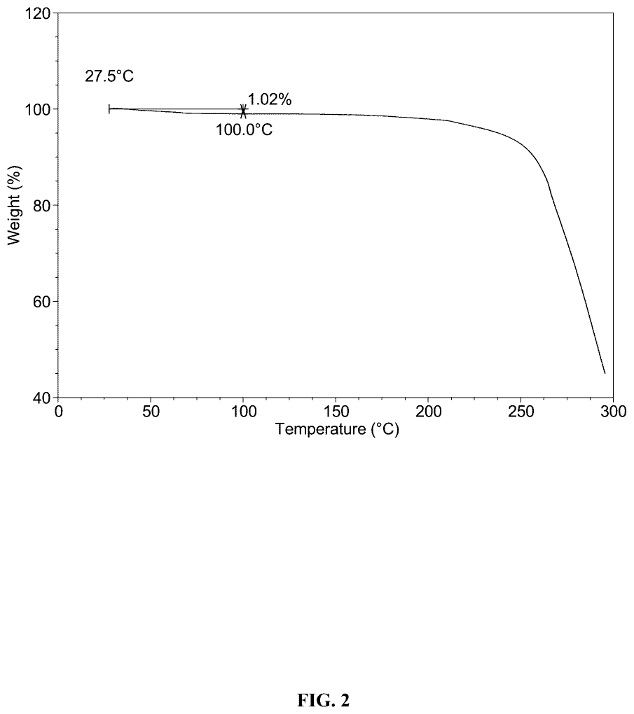

The TGA curve of sample 1 is substantially as depicted in FIG. 2, which shows about 1.0% weight loss when heated to 100° C.

The ¹H NMR data of sample 1 is: H NMR (400 MHz, DMSO-d6) δ 11.29 (s, 1H), 9.30 (brs, 2H), 7.93 (brs, 2H), 7.75 (s, 1H), 7.54 (s, 1H), 7.08 (td, J 9.8, 2.4 Hz, 1H), 6.90 (d, J 9.3 Hz, 1H), 4.22 (s, 1H), 3.51 (s, 2H), 3.30-3.19 (2, 2H), 3.00-2.88 (m, 1H), 2.87-2.69 (m, 2H), 2.61-2.52 (m, 2H), 2.29-2.14 (m, 1H), 2.04-1.73 (m, 3H), 1.64 (d, J=7.6 Hz, 6H), 1.41-1.25 (m, 2H), 0.91 (t, J=7.3 Hz, 3H), 0.85 (s, 9H). The ¹H NMR result shows that Form CSII has no solvent residue.

TABLE 3

| | | Relative |
|---|---|---|
| 2θ (°) | d spacing (Å) | intensity (%) |
| 7.60 | 11.63 | 100.00 |
| 8.08 | 10.95 | 23.97 |
| 8.42 | 10.50 | 96.05 |
| 10.50 | 8.43 | 6.67 |
| 12.00 | 7.38 | 30.35 |
| 13.55 | 6.54 | 7.74 |
| 15.25 | 5.81 | 22.57 |
| 15.70 | 5.64 | 4.14 |
| 16.89 | 5.25 | 11.28 |
| 18.55 | 4.78 | 15.50 |
| 19.48 | 4.56 | 38.13 |
| 20.48 | 4.34 | 18.31 |
| 23.28 | 3.82 | 8.75 |
| 24.14 | 3.69 | 22.14 |
| 24.39 | 3.65 | 16.51 |
| 25.73 | 3.46 | 3.22 |
| 27.17 | 3.28 | 9.04 |
| 30.85 | 2.90 | 15.98 |
| 31.74 | 2.82 | 6.02 |

The sample 2 was confirmed to be Form CSII. The XRPD data are listed in Table 4.

TABLE 4

| | | Relative |
|---|---|---|
| 2θ (°) | d spacing (Å) | intensity (%) |
| 7.60 | 11.63 | 100.00 |
| 8.06 | 10.96 | 21.20 |
| 8.41 | 10.52 | 83.69 |
| 10.51 | 8.42 | 5.59 |
| 12.00 | 7.38 | 30.06 |
| 13.55 | 6.54 | 7.13 |
| 15.25 | 5.81 | 18.83 |
| 15.69 | 5.65 | 3.78 |
| 16.91 | 5.24 | 8.82 |
| 18.54 | 4.78 | 11.44 |
| 19.47 | 4.56 | 33.54 |
| 20.47 | 4.34 | 14.78 |
| 23.27 | 3.82 | 8.42 |
| 23.56 | 3.78 | 6.71 |
| 24.12 | 3.69 | 18.03 |
| 24.44 | 3.64 | 8.99 |
| 25.77 | 3.46 | 3.63 |
| 27.13 | 3.29 | 6.77 |
| 30.81 | 2.90 | 11.93 |
| 31.68 | 2.82 | 5.29 |

### Example 2 Preparation of Form CSIII

201.3 mg of Compound I dihydrobromide was dissolved into 2.0 mL of trifluoroethanol to obtain a clear solution, and then the solution was evaporated at room temperature for 5 days to obtain a solid. The obtained solid was heated to 150° C. under nitrogen protection and the temperature was kept at 150° C. for 5 min, and then the temperature was decreased to room temperature, and then the solid was heated to 210° C. under nitrogen protection and the temperature was kept at 210° C. for 0.1 min, and then the temperature was decreased to room temperature to obtain a crystalline solid.

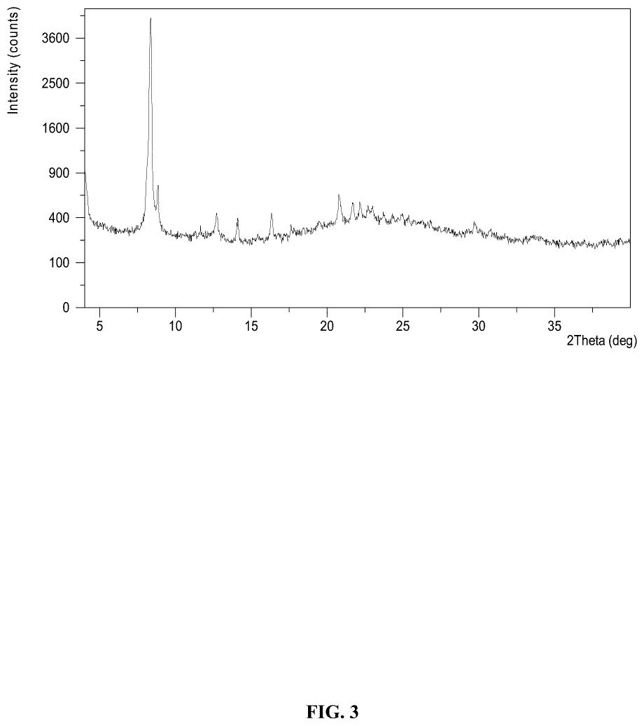

The crystalline solid was confirmed to be Form CSIII. The XRPD pattern of Form CSIII is substantially as depicted in FIG. 3, and the XRPD data are listed in Table 5.

TABLE 5

| | | Relative |
|---|---|---|
| 2θ (°) | d spacing (Å) | intensity (%) |
| 8.36 | 10.58 | 100 |
| 8.84 | 10.01 | 12.01 |
| 12.72 | 6.96 | 5.01 |
| 14.12 | 6.27 | 3.91 |
| 16.34 | 5.43 | 5.12 |
| 20.80 | 4.27 | 7.27 |
| 21.70 | 4.10 | 4.56 |
| 22.18 | 4.01 | 4.83 |
| 23.70 | 3.75 | 1.36 |
| 29.73 | 3.00 | 2.03 |

### Example 3 Preparation of Form CSIII

400.4 mg of Compound I dihydrobromide was added into 18.0 mL of trifluoroethanol to obtain a clear solution, and then the solution was filtered and evaporated at room temperature for 13 days to obtain a solid. The obtained solid was heated to 202° C. under nitrogen protection with a heating rate of 2° C./min, and then the temperature was decreased to room temperature with a cooling rate of 20° C./min to obtain a crystalline solid.

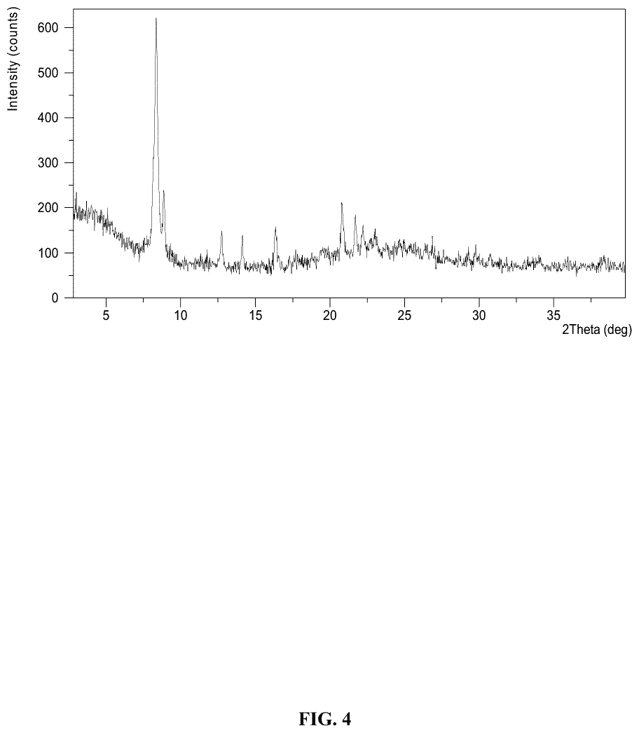

The crystalline solid was confirmed to be Form CSIII. The XRPD pattern of Form CSIII is substantially as depicted in FIG. 4, and the XRPD data are listed in Table 6.

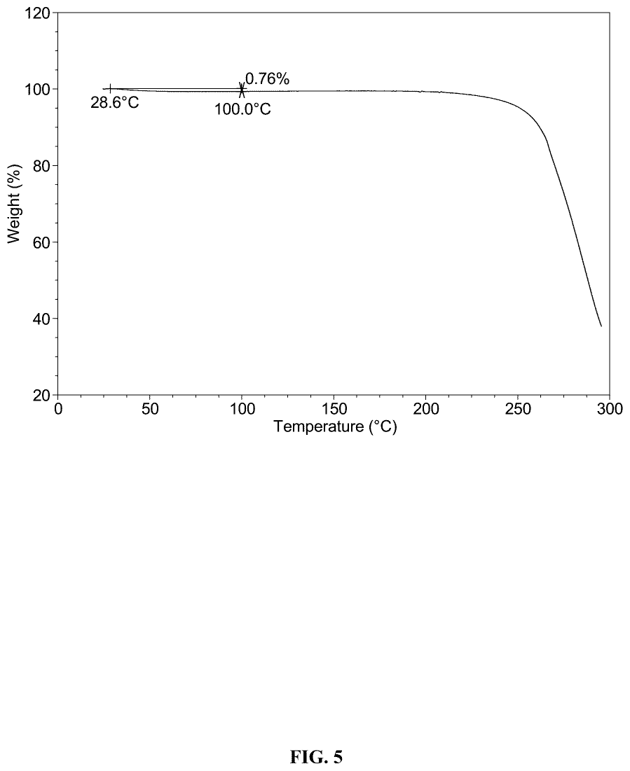

The TGA curve of Form CSIII is substantially as depicted in FIG. 5, which shows about 0.8% weight loss when heated to 100° C.

TABLE 6

| 2θ (°) | d spacing (Å) | Relative intensity (%) |
|---|---|---|
| 8.36 | 10.58 | 100.00 |
| 8.87 | 9.97 | 28.49 |
| 12.74 | 6.95 | 14.38 |
| 14.12 | 6.27 | 12.18 |
| 16.35 | 5.42 | 14.82 |
| 20.80 | 4.27 | 21.09 |
| 21.70 | 4.09 | 14.89 |
| 22.20 | 4.00 | 10.54 |

### Example 4 ¹H NMR Data of Form CSIII

¹H NMR data of Form CSIII is: ¹H NMR (400 MHz, DMSO-d6) δ 11.30 (s, 1H), 9.32 (brs, 2H), 7.95 (brs, 2H), 7.75 (s, 1H), 7.54 (s, 1H), 7.16-7.03 (m, 1H), 6.91 (d, J=9.4 Hz, 1H), 4.22 (s, 1H), 3.51 (s, 2H), 3.32-3.21 (m, 2H), 2.93 (d, J=17.3 Hz, 1H), 2.88-2.63 (m, 2H), 2.63-2.53 (m, 2H), 2.29-2.15 (m, 1H), 1.99-1.74 (m, 3H), 1.63 (d, J=7.8 Hz, 6H), 1.41-1.25 (m, 2H), 0.91 (t, J=7.3 Hz, 3H), 0.85 (s, 9H). The ¹H NMR result shows that Form CSIII has no solvent residue.

### Example 5 Solubility of Form CSII

### Solubility in Water

1.9 mg of Form CSII was weighted into a glass vial, then water was added dropwise at 37° C. until the solid is completely dissolved. The maximum insoluble volume and minimum dissolved volume of the sample in water were recorded, and the solubility of Form CSII in water (mg/mL) was calculated. The results are listed in Table 7. The results show that Form CSII in the present disclosure has higher solubility, and the solubility of Form CSII in water is at least 3-5 times that of Form A.

TABLE 7

| Medium | Form A | Form CSII |
|---|---|---|
| Water | 7 | 19-38 |

The solubility data of Form A is quoted from WO2021029854A1 Example 1.

### Solubility in Biorelevant Medium

When solubility test is used to predict the in vivo performance of a drug, it is critical to simulate in vivo conditions as closely as possible. Fasted state simulated intestinal fluid (FaSSIF) and fed state simulated intestinal fluid (FeSSIF) can be used to simulate the conditions in vivo and predict the effects of eating, thus solubilities in these media are closer to those in vivo.

A certain amount of Form CSII and prior art Form A were suspended into FeSSIF and FaSSIF to get saturated solutions at 37° C. After equilibrated for 1 h, concentrations of the saturated solutions were measured by HPLC. The results are listed in Table 8. The results show that the solubilities of Form CSII are higher. In FeSSIF and FaSSIF, the solubilities of Form CSII are 1.5 times that of prior art Form A.

TABLE 8

| Medium | Form A | Form CSII |
|---|---|---|
| FaSSIF | 6.7 | 9.8 |
| FeSSIF | 7.6 | 11.7 |

### Example 6 Morphology of Form CSII

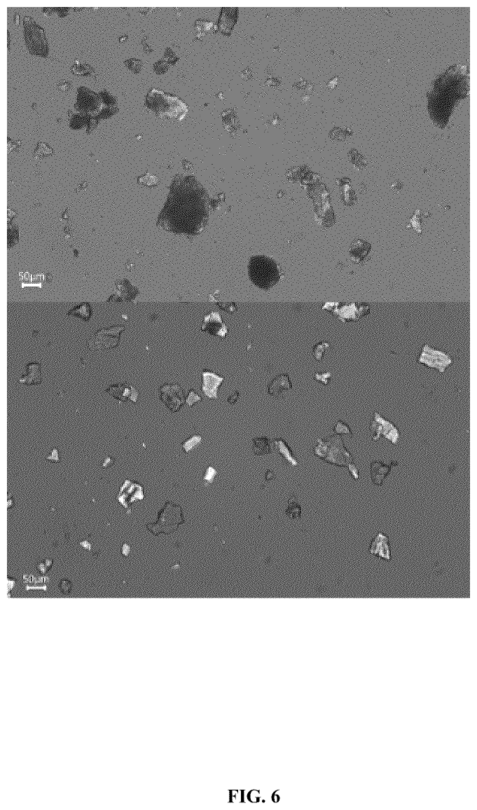

About 0.5 mg Form CSII and prior art Form A were placed on a glass slide, a small amount of immersion oil was added to disperse the sample, and then the system was covered with a coverslip. The morphology of sample was observed with a polarized light microscopy under a magnification of 100 times. The results are shown in FIG. 6. The results show that Form CSII is a uniform plate crystalline form and prior art Form A is agglomerates.

### Example 7 Particle Size Distribution of Form CSII

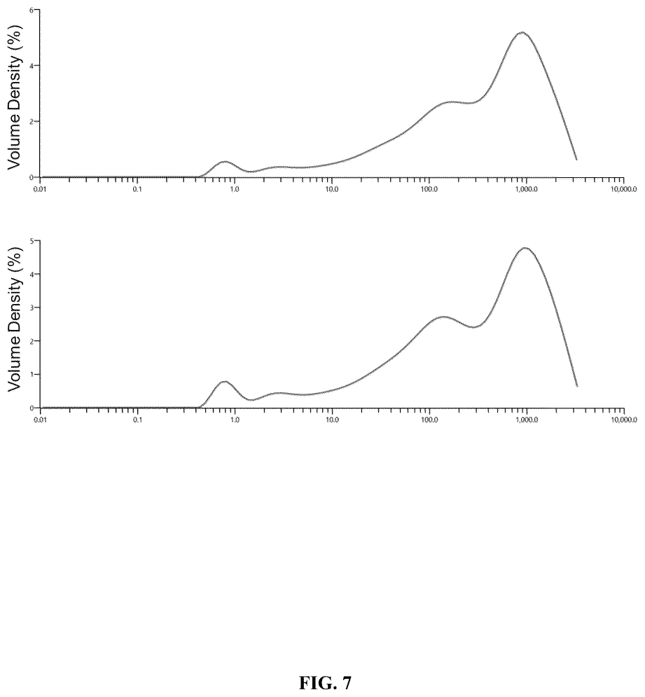

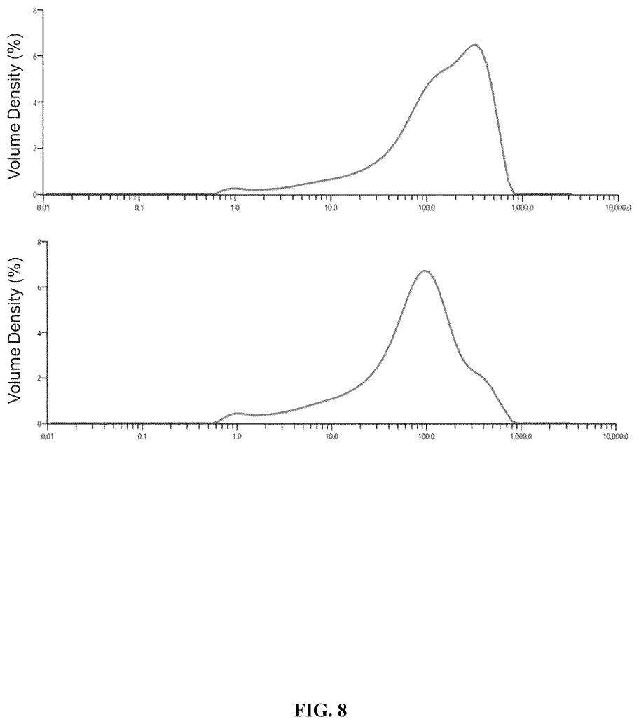

Form CSII and prior art Form A were added into glass vials with the Isopar G (containing 0.2% lecithin). The mixture was mixed thoroughly and transferred into the Hydro MV dispersing device. The experiment was started when the obscuration is in appropriate range. The particle size distributions were tested before and after 60 seconds of ultrasonication to obtain the average particle size, D10 (the portion of particles with diameters below this value is 10%), D50 (the portion of particles with diameters below this value is 50%) and D90 (the portion of particles with diameters below this value is 90%), the results are listed in Table 9. The particle size distribution patterns of Form A in the prior art before and after ultrasonication are shown in FIG. 7. The particle size distribution patterns of Form CSII before and after ultrasonication are shown in FIG. 8. The results shown that there is almost no change in the particle size distribution of prior art Form A before and after ultrasonication, indicating that the particle size distribution of Form A cannot be optimized under external force. Form CSII has a narrow and uniform particle size distribution. Compared with Form A, the particle size of Form CSII is smaller and more uniform.

TABLE 9

| Ultrasonication | | Average particle | D₁₀ | D₅₀ | D₉₀ |
|---|---|---|---|---|---|
| time (s) | Form | size (μm) | (μm) | (μm) | (μm) |
| 0 | Form A | 646 | 21.2 | 415 | 1640 |
| | Form CSII | 198 | 21.9 | 157 | 435 |
| 60 | Form A | 629 | 14.4 | 356 | 1660 |
| | Form CSII | 119 | 10.3 | 81.7 | 282 |

### Example 8 Density and Flowability of Form CSII

About 300-500 mg of powder were added into a 5-mL measuring cylinder, and a bulk density was recorded. Then the powder was tapped for 1250 times by ZS-2E tap density tester to make it in the tightest state and the tapped volume was recorded. The bulk density (ρ₀) and tapped density (ρ_{f}) were calculated.

Compressibility also known as the Compressibility index or Carr index is usually utilized to evaluate the flowability of powder or granules during the drug product process. Compressibility index was calculated according to c=(ρ_{f}−ρ₀)/ρ_{f}. According to criteria of flowability recorded in ICH Q4B Annex 13, the smaller the compressibility index, the better the flowability.

Flowability evaluation results of Form CSII and prior art Form A are listed in Table 10, which indicate that flowability of Form CSII is remarkably superior to that of prior art Form A.

TABLE 10

| | Bulk density | Tapped density | Compressibility |
|---|---|---|---|
| Form | (ρ₀, g/mL) | (ρ_{f}, g/mL) | index (%) |
| Form A | 0.251 | 0.282 | 10.8% |
| Form CSII | 0.49 | 0.51 | 5.3% |

### Example 9 Compressibility of Form CSII

ENERPAC manual tablet press was used for compression. 60 mg of Form CSII and prior art Form A were weighed and added into the dies of a Φ6 mm round tooling, compressed at 3 KN manually, then stored at room temperature for 24 h until elastic recovery is complete, diameter (D) and thickness (L) were tested with a caliper. Hardness (H) was tested with an intelligent tablet hardness tester. Tensile strength of the powder was calculated with the following formula: T=2H/πDL. The results shown that the tensile strength of Form CSII is 0.68 MPa and the tensile strength of Form A is 0.37 MPa. Under a certain force, the greater the tensile strength, the better the compressibility. Therefore, the results indicate that Form CSII has better compressibility compared with Form A.

### Example 10 Physical Stability of Form CSII Upon Mechanical Force

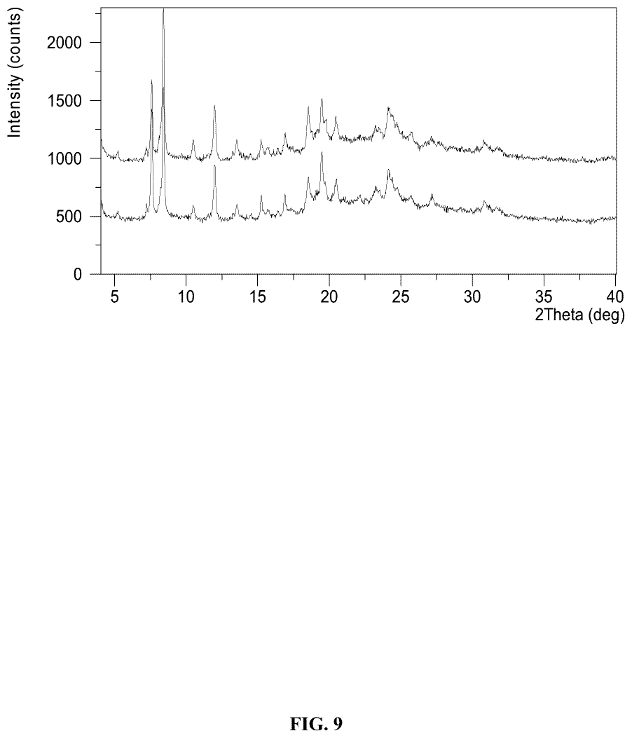

A certain amount of Form CSII was compressed into a tablet under 5 kN pressure with the dies of a Φ6 mm round tooling by the manual tablet press, and the tablet was kept under 5 kN pressure for 10 s. Crystalline form before and after tableting were checked by XRPD. The test results are shown in FIG. 9 and the results show that Form CSII remains stable after tableting.

### Example 11 Stability of Form CSII

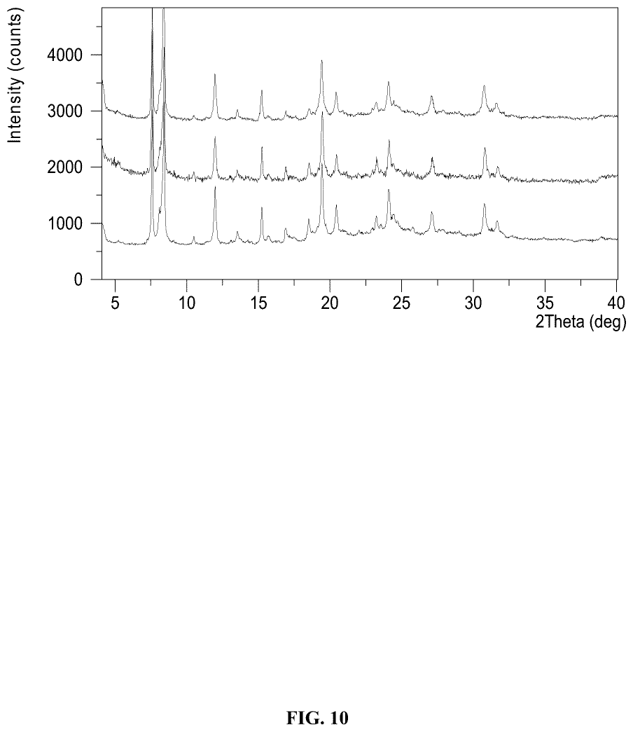

A certain amount of Form CSII were stored under different conditions of 25° C./60% RH and 40° C./75% RH after packing with different conditions listed in Table 11. Crystalline forms and chemical purities were checked by XRPD and HPLC, respectively. The results are shown in Table 11, and the XRPD overlay is shown in FIG. 10. The results indicate that Form CSII sealed with desiccant and antioxidant can keep stable for at least 9 months at 25° C./60% RH and at least 6 months 40° C./75% RH. Form CSII has good stability under both long-term and accelerated conditions.

TABLE 11

| Initial | | | | | |
|---|---|---|---|---|---|
| form | Condition | Packing condition | Time | Form | Purity |
| Form | initial | / | / | Form | 99.37% |
| CSII | | | | CSII | |
| | 25° C./ | Sealed with desiccants | 9 | Form | 99.43% |
| | 60% RH | and antioxidants | months | CSII | |
| | 40° C./ | Sealed with desiccants | 6 | Form | 99.42% |
| | 75% RH | and antioxidants | months | CSII | |

Sealed with desiccants and antioxidants: Put a sample into a glass vial, cover the vial with aluminum foil, and punch holes in the foil, then seal the glass vial with 2 g silica gel desiccants and 2.2 g antioxidants in an aluminum foil bag.

### Example 12 Preparation of Form CSII Drug Product

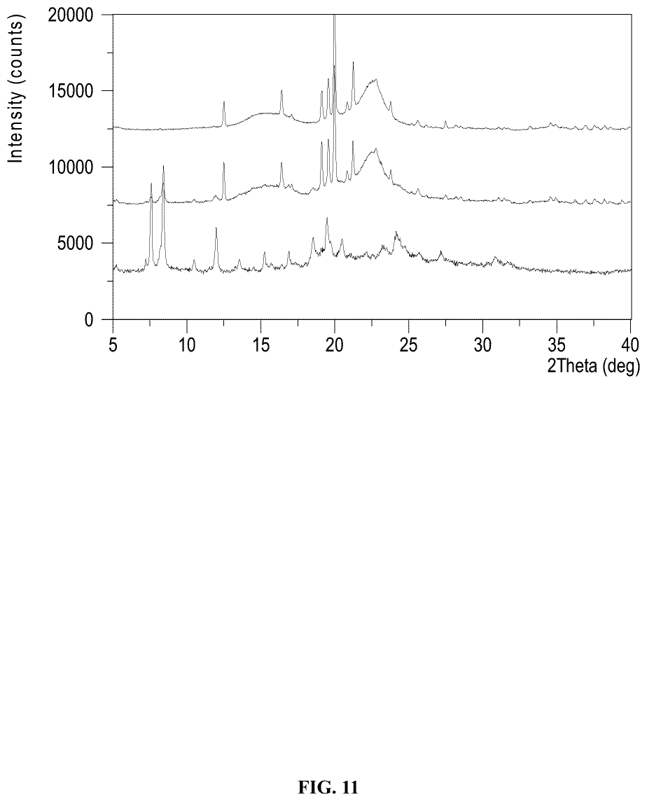

The formulation of Form CSII and the blank mixed powder are shown in Table 12 and Table 13. The preparation process of Form CSII is shown in Table 14. The XRPD of the blank mixed powder and the samples before and after the formulation was tested. The results are showed in FIG. 11 and indicate that Form CSII remains stable after the formulation process.

TABLE 12

| | Component | % (w/w) | mg/unit |
|---|---|---|---|
| 1 | Compound I dihydrobromide | 26.6 | 26.6 |
| 2 | Microcrystalline Cellulose | 51.4 | 51.4 |
| 3 | Lactose | 16 | 16 |
| 4 | Crospovidone | 5 | 5 |
| 5 | Magnesium stearate | 0.5 | 0.5 |
| 6 | Microsilica gel | 0.5 | 0.5 |

| | |
|---|---|
| Total | 100 |

Note:
26.6 mg Compound I dihydrobromide is corresponding to 20 mg Compound I free base.

TABLE 13

| | | |
|---|---|---|
| Blank formulation | % (w/w) | Function |

| | | | |
|---|---|---|---|
| 1 | Microcrystalline Cellulose | 70.0 | Filler |
| 2 | Lactose | 21.8 | Filler |
| 3 | Crospovidone | 6.8 | Disintegrant |
| 4 | Magnesium stearate | 0.7 | Lubricant |
| 5 | Microsilica gel | 0.7 | Glidant |

| | | |
|---|---|---|
| Total | 100.0 | / |

TABLE 14

| Stage | Procedure |
|---|---|
| Pre-blending | According to the formulation, materials No. 1-6 |
| | were weighed into an LDPE bags and blended for |
| | 2 mins. |
| Simulation of | The pre-blended powder was pressed by a single |
| dry granulation | punch manual tablet press (type: ENERPAC; die: |
| | ϕ20 mm round; flake weight: 500 mg ± 10 mg; |
| | pressure: 5 ± 1 kN) to obtain a flake. The |
| | flake was pulverized and sieved through a 20- |
| | mesh sieve to obtain the final blended powder. |
| Tableting | The final blended powder was tableted by a |
| | single punch manual tablet press (die: 9 × 4 |
| | mm; pressure: 5 kN) |
| Packing | Seal a tablet with 1 g antioxidant and 2 g |
| | desiccant in a 35 cc HDPE bottle. |

### Example 13 Stability of Form CSII in Drug Product

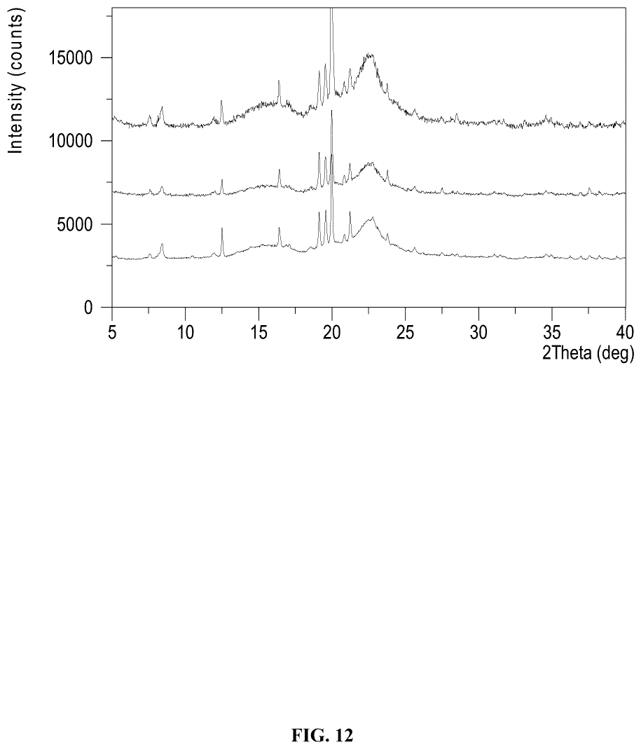

The tablets of Form CSII were packed with 1 g antioxidants and 2 g desiccants and stored under 40° C./75% RH and 60° C./75% RH conditions for one month. Crystalline forms were checked by XRPD, and the results are shown in FIG. 12. The results indicate that Form CSII drug product can keep stable under 40° C./75% RH and 60° C./75% RH for at least one month.

### Example 14 Morphology of Form CSIII

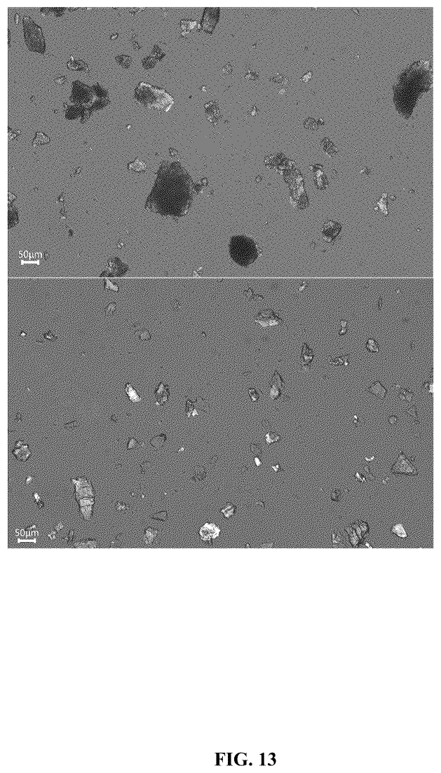

About 0.5 mg Form CSIII and prior art Form A were placed on a glass slide, a small amount of immersion oil was added to disperse the sample, and then the system was covered with a coverslip. The morphology of sample was observed with a polarized light microscopy under a magnification of 100 times. The results are shown in FIG. 13. The results show that Form CSIII is a uniform plate crystalline form and prior art Form A is agglomerates.

### Example 15 Solubility of Form CSIII

When solubility test is used to predict the in vivo performance of a drug, it is critical to simulate in vivo conditions as closely as possible. Fasted state simulated intestinal fluid (FaSSIF) and fed state simulated intestinal fluid (FeSSIF) can be used to simulate the conditions in vivo and predict the effects of eating, thus solubilities in these media are closer to those in vivo.

A certain amount of Form CSIII and Form A in the prior art were suspended into FeSSIF and FaSSIF to get saturated solutions at 37° C. After equilibrated for 1 h, concentrations of the saturated solutions were measured by HPLC. The results are listed in Table 15. The results show that the solubilities of Form CSIII are higher than prior art. In FaSSIF, the solubility of Form CSIII is 1.3 times that of prior art Form A. In FeSSIF, the solubility of Form CSIII is 1.5 times that of prior art Form A in.

TABLE 15

| Medium | Form A | Form CSIII |
|---|---|---|
| FaSSIF | 6.7 | 8.7 |
| FeSSIF | 7.6 | 11.7 |

### Example 16 Stability of Form CSIII

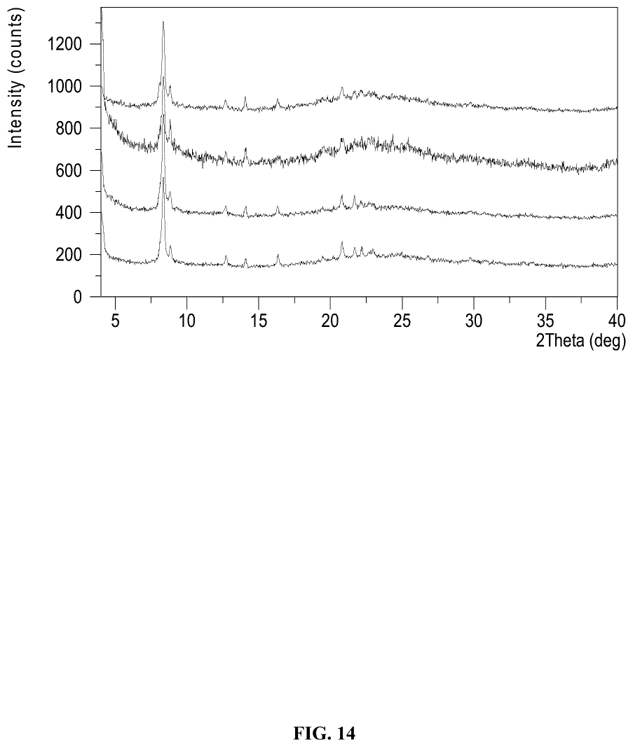

A certain amount of Form CSIII were stored under different conditions of 25° C./60% RH, 40° C./75% RH and 60° C./75% RH after packing with different conditions listed in Table 16. Crystalline forms were checked by XRPD. The results are shown in Table 16, and the XRPD overlay is shown in FIG. 14. The results indicate that Form CSIII sealed with desiccants and antioxidants can keep stable for at least 3 months at 25° C./60% RH and 40° C./75% RH, as well as at least 2 months at 60° C./75% RH. Form CSIII has good chemical stability under long-term, accelerated and stress conditions.

TABLE 16

| Initial form | Condition | Packing condition | Time | Form |
|---|---|---|---|---|
| Form CSIII | initial | / | / | Form |
| | | | | CSIII |
| | 25° C./ | Sealed with desiccants | 3 months | Form |
| | 60% RH | and antioxidants | | CSIII |
| | 40° C./ | Sealed with desiccants | 3 months | Form |
| | 75% RH | and antioxidants | | CSIII |
| | 60° C./ | Sealed with desiccants | 2 months | Form |
| | 75% RH | and antioxidants | | CSIII |

Sealed with desiccants and antioxidants: Put a sample into a glass vial, cover the vial with aluminum foil, and punch holes in the foil, then seal the glass vial with 2 g silica gel desiccant and 2.2 g antioxidant in an aluminum foil bag.

### Example 17 Preparation of Solvate Type K13

0.3 mL of the corresponding solvent listed in Table 17 was added to a certain amount of Compound I dihydrobromide listed in Table 17, and then the system was shook at −20° C. for 3-23 hours to obtain Type K13. Inventors of the present disclosure found that Type K13 is isomorphous and can be toluene solvate, anisole solvate, trifluoroethanol solvate, methyl isobutylketone solvate, co-solvate of toluene and anisole, co-solvate of methyl tert-butyl ether and anisole, co-solvate of toluene and trifluoroethanol, and co-solvate of toluene and methyl tert-butyl ether.

TABLE 17

| | Weight | | |
|---|---|---|---|
| No. | (mg) | Solvent (v/v) | Volume (mL) |
| 1 | 9.3 | Methyl isobutyl ketone | 0.3 |
| 2 | 9.8 | Anisole | 0.3 |
| 3 | 9.1 | Toluene/Anisole (1:1) | 0.3 |
| 4 | 9.8 | Methyl tert-butyl ether/Anisole (1:1) | 0.3 |

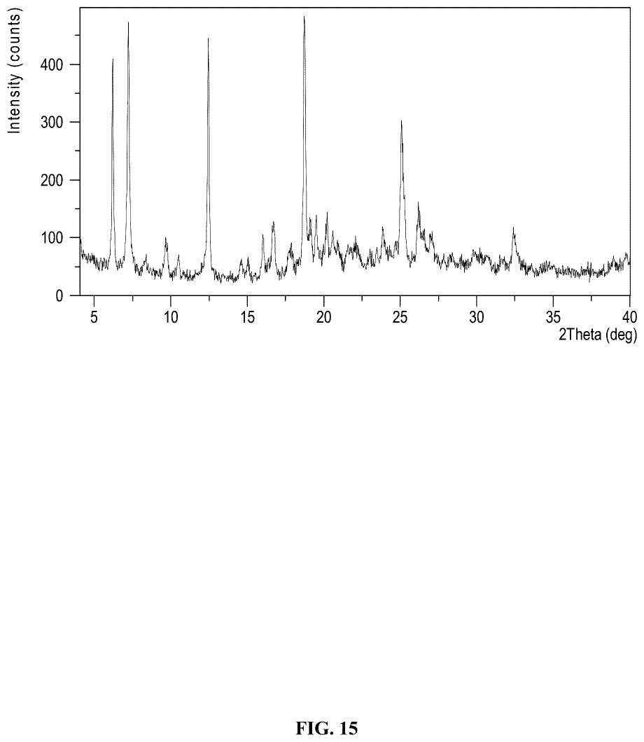

The XRPD pattern of Type K13 is substantially as depicted in FIG. 15, and the XRPD data are listed in Table 18.

TABLE 18

| | | Relative |
|---|---|---|
| 2θ (°) | d spacing (Å) | intensity (%) |
| 6.2 | 14.3 | 82.2 |
| 7.2 | 12.2 | 91.6 |
| 8.3 | 10.6 | 4.5 |
| 9.6 | 9.2 | 10.7 |
| 10.5 | 8.4 | 8.3 |
| 12.5 | 7.1 | 93.9 |
| 14.6 | 6.1 | 7.3 |
| 15.0 | 5.9 | 5.7 |
| 16.0 | 5.5 | 15.8 |
| 16.4 | 5.4 | 6.8 |
| 16.8 | 5.3 | 18.0 |
| 17.9 | 5.0 | 10.9 |
| 18.7 | 4.7 | 100.0 |
| 19.1 | 4.6 | 22.2 |
| 19.5 | 4.6 | 24.4 |
| 20.2 | 4.4 | 22.1 |
| 20.6 | 4.3 | 16.5 |
| 21.0 | 4.2 | 10.4 |
| 21.5 | 4.1 | 11.3 |
| 22.1 | 4.0 | 11.5 |
| 23.0 | 3.9 | 8.4 |
| 23.5 | 3.8 | 10.9 |
| 23.9 | 3.7 | 18.1 |
| 24.7 | 3.6 | 12.7 |
| 25.1 | 3.6 | 58.6 |
| 26.2 | 3.4 | 21.7 |
| 26.6 | 3.4 | 15.9 |
| 27.0 | 3.3 | 14.7 |
| 27.9 | 3.2 | 7.0 |
| 28.4 | 3.1 | 7.2 |
| 29.8 | 3.0 | 9.4 |
| 30.7 | 2.9 | 6.8 |
| 31.7 | 2.8 | 5.1 |
| 32.4 | 2.8 | 16.7 |
| 33.5 | 2.7 | 2.4 |
| 35.1 | 2.6 | 2.2 |
| 38.9 | 2.3 | 6.4 |
| 39.4 | 2.3 | 3.5 |
| 39.8 | 2.3 | 6.5 |

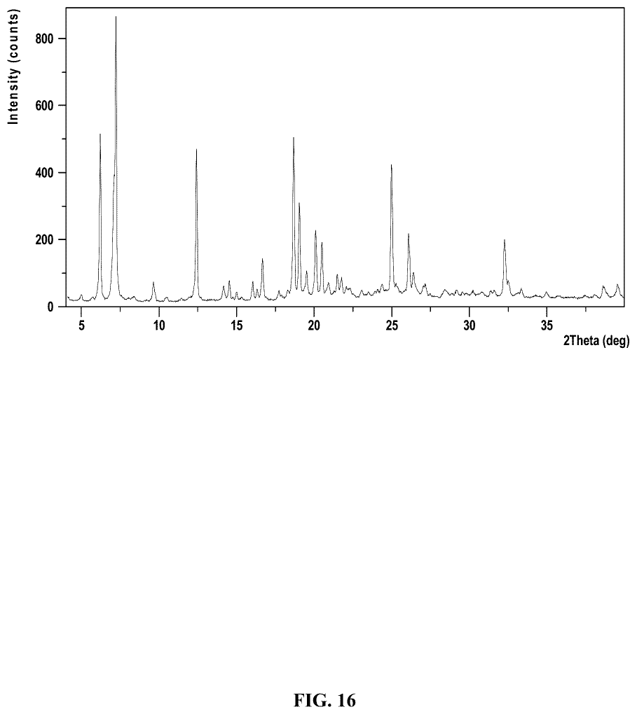

The XRPD pattern of Type K13 is substantially as depicted in FIG. 16, and the XRPD data are listed in Table 19.

TABLE 19

| | | Relative |
|---|---|---|
| 2θ (°) | d spacing (Å) | intensity (%) |
| 5.0 | 17.8 | 1.8 |
| 6.2 | 14.3 | 41.9 |
| 7.2 | 12.2 | 100.0 |
| 8.4 | 10.5 | 1.7 |
| 9.7 | 9.2 | 6.6 |
| 10.5 | 8.4 | 1.2 |
| 11.5 | 7.7 | 0.9 |
| 12.4 | 7.1 | 52.9 |
| 14.1 | 6.3 | 3.9 |
| 14.6 | 6.1 | 4.9 |
| 15.0 | 5.9 | 2.3 |
| 15.3 | 5.8 | 1.0 |
| 16.0 | 5.5 | 4.6 |
| 16.7 | 5.3 | 14.6 |
| 17.8 | 5.0 | 2.4 |
| 18.7 | 4.8 | 45.6 |
| 19.1 | 4.7 | 26.6 |
| 20.1 | 4.4 | 24.8 |
| 20.5 | 4.3 | 19.5 |
| 20.9 | 4.3 | 5.9 |
| 21.6 | 4.1 | 4.1 |
| 22.2 | 4.0 | 3.6 |
| 23.1 | 3.9 | 3.2 |
| 24.4 | 3.7 | 4.7 |
| 25.0 | 3.6 | 41.1 |
| 26.1 | 3.4 | 19.9 |
| 27.2 | 3.3 | 5.0 |
| 28.4 | 3.1 | 2.9 |
| 29.2 | 3.1 | 3.1 |
| 30.2 | 3.0 | 2.6 |
| 30.8 | 2.9 | 2.3 |
| 31.5 | 2.8 | 2.2 |
| 32.3 | 2.8 | 18.7 |
| 33.3 | 2.7 | 2.7 |
| 34.2 | 2.6 | 0.8 |
| 35.0 | 2.6 | 2.1 |
| 35.8 | 2.5 | 0.6 |
| 37.5 | 2.4 | 0.8 |
| 38.1 | 2.4 | 1.2 |
| 38.6 | 2.3 | 4.3 |
| 39.6 | 2.3 | 4.4 |

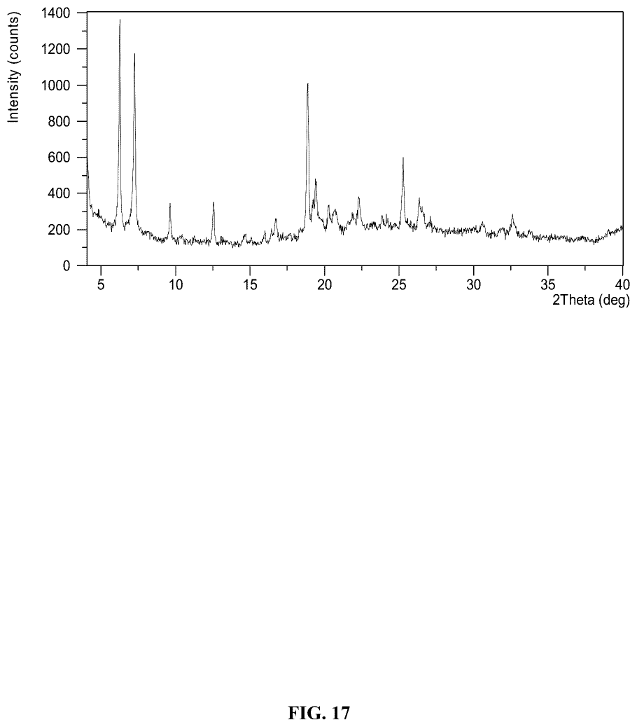

The XRPD pattern of Type K13 is substantially as depicted in FIG. 17, and the XRPD data are listed in Table 20.

TABLE 20

| | | Relative |
|---|---|---|
| 2θ (°) | d spacing (Å) | intensity (%) |
| 6.3 | 14.1 | 100.0 |
| 7.2 | 12.2 | 85.9 |
| 9.6 | 9.2 | 16.3 |
| 12.5 | 7.1 | 20.3 |
| 14.6 | 6.0 | 4.2 |
| 16.0 | 5.6 | 4.3 |
| 16.7 | 5.3 | 10.9 |
| 18.9 | 4.7 | 78.4 |
| 19.4 | 4.6 | 27.2 |
| 20.3 | 4.4 | 15.6 |
| 20.7 | 4.3 | 14.2 |
| 22.3 | 4.0 | 19.6 |
| 25.3 | 3.5 | 37.8 |
| 26.4 | 3.4 | 17.3 |
| 26.7 | 3.3 | 8.1 |
| 30.6 | 2.9 | 4.9 |
| 32.6 | 2.7 | 10.4 |

The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

What is claimed is:

1. A crystalline form CSII of Compound I dihydrobromide, wherein the X-ray powder diffraction pattern comprises characteristic peaks at 2theta values of 7.6°±0.2°, 8.4°±0.2° and 12.0°±0.2° using CuKα radiation,

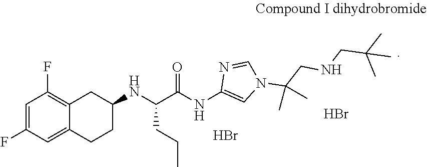 [SMILES: CCC[C@H](N[C@H]1CCc2cc(F)cc(F)c2C1)C(=O)Nc1cn(C(C)(C)CNCC(C)(C)C)cn1 ; Br ; Br]

2. The crystalline form CSII of Compound I dihydrobromide according to claim 1, wherein the X-ray powder diffraction pattern comprises one or two or three characteristic peaks at 2theta values of 18.5°±0.2°, 19.5°±0.2° and 20.5°±0.2° using CuKα radiation.

3. The crystalline form CSII of Compound I according to claim 2, wherein the X-ray powder diffraction pattern is substantially as depicted in FIG. 1 using CuKα radiation.

4. A pharmaceutical composition, wherein said pharmaceutical composition comprises a therapeutically effective amount of crystalline form CSII of Compound I dihydrobromide according to claim 1, and pharmaceutically acceptable excipients.

5. A method of preparing drugs of γ-secretase inhibitor, comprising using crystalline form CSII of Compound I dihydrobromide according to claim 1.

6. A method of preparing drugs for treating desmoid tumors, comprising using crystalline form CSII of Compound I dihydrobromide according to claim 1.
